(19) United States
(12) Patent Application Publication
(10) Pub. No.: US 2025/0199573 A1
(43) Pub. Date: Jun. 19, 2025

(54) NETWORK CONTROLLED OPERATION OF WEARABLE DEVICE

(71) Applicant: Telefonaktiebolaget LM Ericsson (publ), Stockholm (SE)

(72) Inventors: Alexey SHAPIN, Luleå (SE); Luis Felipe DEL CARPIO VEGA, Espoo (FI); Dhruvin PATEL, Aachen (DE); Ajay KATTEPUR, Bangalore (IN); Rong DU, Bromma (SE)

(21) Appl. No.: 18/838,054

(22) PCT Filed: Feb. 14, 2022
(86) PCT No.: PCT/SE2022/050159

§ 371 Date: Aug. 13, 2024

(51) Int. Cl. G06F 1/16; G06F 3/01; H04W 76/27

(52) U.S. Cl. CPC G06F 1/163; G06F 3/013; H04W 76/27

(57) ABSTRACT

Embodiments of the present disclosure provide a method for controlling an operation of a wearable device that is connected to a network node in a wireless communication network. The method is performed by the wearable device. The method includes identifying an activity status for user activity using the wearable device and providing information including at least one parameter associated with the wearable device and the identified activity status. The method includes transmitting the provided information to the network node in the wireless communication network and receiving, from the network node, control information including at least one indication for controlling the operation of the wearable device. The method further includes controlling the operation of the wearable device in accordance with the control information received from the network node. Corresponding wearable device, network node and computer program products are also disclosed.

### TECHNICAL FIELD

The present disclosure relates generally to wearable devices. More particularly, it relates to methods, wearable device, network node, and computer program products for controlling an operation of a wearable device.

### BACKGROUND

Portable electronic devices such as smart phones, wearable devices and other electronic devices have become an everyday need in the way we communicate and interact with others. The frequent use of these devices may require a significant amount of power, which may easily deplete the batteries attached to these devices. In one example, a wearable device may be capable of executing extended reality, XR, function to render a content on a screen of the wearable device. The XR encompasses augmented reality, AR, virtual reality, VR, mixed reality, MR, or the like. The AR enhances our view of the real world by overlaying what we see with computer-generated information, while the VR completely replaces a user's view, immersing the user within a computer-generated virtual environment. The MR merges the real world and virtual world. The XR is used to denote the concept of presenting a combination of real-world objects and computer-rendered content to a user.

A wearable device having XR capability generally receives content from a content provider through a wireless communication network. Further, the wearable device may render the content to a user. The wearable device utilizes battery power for receiving and rendering the content to the user. Thus, there can be significant impact on processing load and battery power consumption of wearable device. Apart from processing load and battery power consumption at the wearable device, the transfer of total amount of data bits over an often limited/constrained communication interface (e.g. over air interface) consumes radio resources, which may lead to increase in usage of radio resources with emerging use of XR capable wearable devices.

Consequently, there is a need to optimize the usage of the radio resources along with power consumption of wearable devices.

### SUMMARY

Consequently, there is a need for an improved method and arrangement for optimization of power consumption of wearable devices that alleviates at least some of the above cited problems.

It is therefore an object of the present disclosure to provide a method, a wearable device, a network node and a computer program product for optimization of power consumption of wearable devices to mitigate, alleviate, or eliminate all or at least some of the above-discussed drawbacks of presently known solutions.

This and other objects are achieved by means of a method, a wearable device, a network node, and a computer program product as defined in the appended claims. The term exemplary is in the present context to be understood as serving as an instance, example or illustration.

According to a first aspect of the present disclosure, a method for controlling an operation of a wearable device is disclosed. The wearable device is connected to a network node in a wireless communication network. The method is performed by the wearable device. The method comprises identifying an activity status for user activity using the wearable device and providing information comprising at least one parameter associated with the wearable device and the identified activity status. The method comprises transmitting the provided information to the network node in the wireless communication network and receiving, from the network node, control information comprising at least one indication for controlling the operation of the wearable device. The method further comprises controlling the operation of the wearable device in accordance with the control information received from the network node.

In some embodiments, the method further comprises obtaining the activity status and/or the at least one parameter associated with the wearable device from one or more sensors equipped in the wearable device.

In some embodiments, the at least one parameter associated with the wearable device comprises one or more of: a battery level of the wearable device, a position of the wearable device, a movement of the wearable device, an orientation of the wearable device, and one or more applications executing at the wearable device.

In some embodiments, the activity status comprises one or more of: information whether the wearable device being worn by the user or not, an eye gaze movement pattern of the user on a screen of the wearable device, a velocity with which the user is moving while wearing the wearable device, conversation of the user while wearing the wearable device, presence of another user in proximity to the user, and an eye gaze direction of the user on the wearable device.

In some embodiments, the control information comprises one or more of: an indication to change a Radio Resource Control, RRC, state of the wearable device, a change in an allocation of radio resources for the wearable device, an indication to change in video encoding parameters associated with the wearable device in accordance with the activity status using the wearable device, an indication for monitoring of a Physical Downlink Control Channel, PDCCH, information, and an indication for a RRC connection reconfiguration of the wearable device.

According to a second aspect of the present disclosure, a method for controlling an operation of a wearable device is disclosed. The wearable device is connected to a network node in a wireless communication network. The method is performed by the network node. The method comprises receiving, from the wearable device, information comprising at least one parameter associated with the wearable device and an activity status for user activity using the wearable device. The method comprises providing control information for control of the wearable device. The control information is based on the information received from the wearable device. The method further comprises transmitting, to the wearable device, the provided control information comprising at least one indication for controlling the operation of the wearable device.

In some embodiments, at least a part of the information is received from one or more sensors equipped in the wearable device.

In some embodiments, the at least one parameter associated with the wearable device comprises one or more of: a battery level of the wearable device, a position of the wearable device, a movement of the wearable device, an orientation of the wearable device, and one or more applications executing at the wearable device.

In some embodiments, the activity status comprises one or more of: information whether the wearable device being worn by the user or not, an eye gaze movement pattern of the user on a screen of the wearable device, a velocity with which the user is moving while wearing the wearable device, conversation of the user while wearing the wearable device, presence of another user in proximity to the user, and an eye gaze direction of the user on the wearable device.

In some embodiments, the control information comprises one or more of: an indication to change a Radio Resource Control, RRC, state of the wearable device, a change in an allocation of radio resources for the wearable device, an indication to change in video encoding parameters associated with the wearable device in accordance with the activity status using the wearable device, an indication for monitoring of a Physical Downlink Control Channel, PDCCH, information, and an indication for a RRC connection reconfiguration of the wearable device.

According to a third aspect of the present disclosure, a wearable device for controlling an operation of the wearable device is provided. The wearable device is connected to a network node in the wireless communication network. The wearable device comprising controlling circuitry configured to cause identification of an activity status for user activity using the wearable device and providing of information comprising at least one parameter associated with the wearable device and the identified activity status. The controlling circuitry is configured to cause transmission of the provided information to the network node in the wireless communication network and reception of control information from the network node, the control information comprising at least one indication for controlling the operation of the wearable device. Further, the controlling circuitry is configured to cause controlling of the operation of the wearable device in accordance with the control information received from the network node.

According to a fourth aspect of the present disclosure, an apparatus of a network node configured to operate in a wireless communication network for controlling an operation of the wearable device is provided. The wearable device is connected to a network node in the wireless communication network. The apparatus comprising controlling circuitry configured to cause reception of information from the wearable device, the information comprising at least one parameter associated with the wearable device and an activity status for user activity using the wearable device. The controlling circuitry is configured to cause providing of control information for control of the wearable device. The control information is based on the information received from the wearable device. Further, the controlling circuitry is configured to cause transmission of the provided control information to the wearable device, the control information comprising at least one indication for controlling an operation of the wearable device.

A fifth aspect is a network node comprising the apparatus of the fifth aspect.

According to a sixth aspect of the present disclosure, there is provided a computer program product comprising a non-transitory computer readable medium, having thereon a computer program comprising program instructions. The computer program is loadable into a data processing unit and configured to cause execution of the method according to the first and second aspects when the computer program is run by the data processing unit.

In some embodiments, any of the above aspects may additionally have features identical with or corresponding to any of the various features as explained above for any of the other aspects.

An advantage of some embodiments is that alternative and/or improved approaches are provided for controlling the operation of the wearable device using an activity status for user activity and parameter(s) associated with wearable device.

An advantage of some embodiments is that the efficiency of radio resource usage is increased in the wireless communication network.

An advantage of some embodiments is that the power consumption at the wearable device is optimized.

### BRIEF DESCRIPTION OF THE DRAWINGS

The foregoing will be apparent from the following more particular description of the example embodiments, as illustrated in the accompanying drawings in which like reference characters refer to the same parts throughout the different views. The drawings are not necessarily to scale, emphasis instead being placed upon illustrating the example embodiments.

FIG. 1 discloses an example environment according to some embodiments;

FIG. 2 is a flowchart illustrating example method steps according to some embodiments;

FIG. 3 is a flowchart illustrating example method steps according to some embodiments;

FIGS. 4A, 4B, and 4C are example illustrations depicting an activity status for user activity using the wearable device according to some embodiments;

FIG. 5 is a block diagram of a wearable device and a network node according to some embodiments;

FIG. 6 discloses an example computing environment according to some embodiments;

FIG. 7 is a block diagram of a telecommunication network connected via an intermediate network to a host computer, according to some embodiments;

FIG. 8 is a block diagram of a host computer communicating via a base station with a UE over a partially wireless connection, according to some embodiments;

FIG. 9 is a block diagram of methods implemented in a communication system including a host computer, a base station, and a UE, according to some embodiments;

FIG. 10 is a block diagram of methods implemented in a communication system including a host computer, a base station, and a UE, according to some embodiments;

FIG. 11 is a block diagram of methods implemented in a communication system including a host computer, a base station, and a UE, according to some embodiments; and

FIG. 12 is a block diagram of methods implemented in a communication system including a host computer, a base station, and a UE, according to some embodiments.

### DETAILED DESCRIPTION

Aspects of the present disclosure will be described more fully hereinafter with reference to the accompanying drawings. The apparatus and methods disclosed herein can, however, be realized in many different forms and should not be construed as being limited to the aspects set forth herein. Like numbers in the drawings refer to like elements throughout.

The terminology used herein is for the purpose of describing particular aspects of the disclosure only and is not intended to limit the invention. It should be emphasized that the term “comprises/comprising” when used in this specification is taken to specify the presence of stated features, integers, steps, or components, but does not preclude the presence or addition of one or more other features, integers, steps, components, or groups thereof. As used herein, the singular forms “a”, “an” and “the” are intended to include the plural forms as well, unless the context clearly indicates otherwise.

Embodiments of the present disclosure will be described and exemplified more fully hereinafter with reference to the accompanying drawings. The solutions disclosed herein can, however, be realized in many different forms and should not be construed as being limited to the embodiments set forth herein.

It will be appreciated that when the present disclosure is described in terms of a method, it may also be embodied in one or more processors and one or more memories coupled to the one or more processors, wherein the one or more memories store one or more programs that perform the steps, services and functions disclosed herein when executed by the one or more processors.

In the present disclosure, wearable device is enabled to communicate wirelessly with a network node in a wireless communication network.

Typically, the network node serves or covers one or several cells of the wireless communication network. That is, the network node provides radio coverage in the cell(s) and communicates over an air interface with the UE(s) operating on radio frequencies within its range. The network node may be also referred to as “eNB”, “eNodeB”, “NodeB” or “gNB”, depending on the technology and terminology used. In the present disclosure, the network node device may also be referred to as a base station, BS.

In the present disclosure, it is assumed that connection establishment has already been completed between the UE and the network node.

In the following description of exemplary embodiments, the same reference numerals denote the same or similar components.

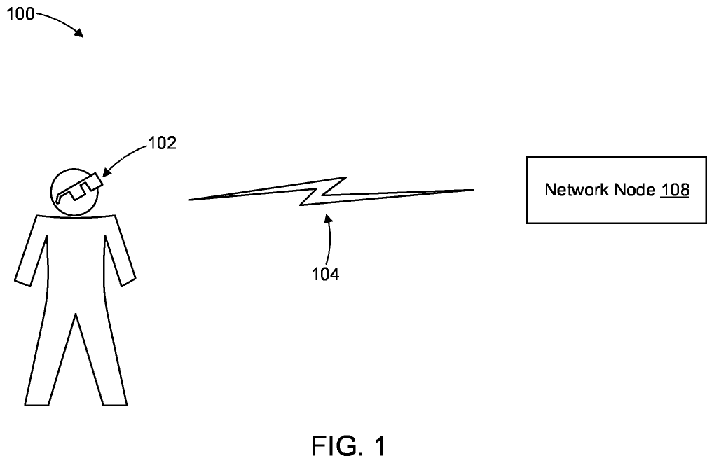

FIG. 1 discloses an example environment 100 of operation of a wearable device 102. A user wears the wearable device 102. The wearable device 102 may comprise a head mounted display (HMD). The HMD is head-mounted display device mounted on a user's head to view an image or a content which is emerging. The HMD is mounted on a part of a body, for example, a head, of a user to display the content implementing extended reality, XR. Such a HMD device 10 has a goggle shape or a glasses shape, as shown in FIG. 1. The wearable device 102 is connected to a network node 108 through a wireless link 104. For example, the wearable device 102 obtains the content from the network node 108 through the wireless communication network.

The wearable device 102 (i.e., the HMD), allows the user to see both real-world objects and computer-rendered content. Real-world objects are physical objects which are not computer-rendered, i.e., not virtual. For instance, the HMD can be implemented using extended reality (XR) glasses such as Microsoft Hololens. It is to be noted that the term ‘computer’ used herein is to be interpreted as any electronic device with digital processing capabilities, regardless of size. Hence, the term computer comprises devices such as servers, desktop computers, laptop computers, tablet computers, smart phones, mobile phones, smart watches, etc.

The wearable device 102 consumes battery power for receiving and rendering the content. Thus, there can be significant impact on processing load and battery power consumption of wearable device 102. Apart from the processing load and the battery power consumption at the wearable device 102, the transfer of total amount of data bits over an often limited/constrained communication interface (e.g. over air interface) consumes radio resources, which may lead to increase in usage of radio resources with emerging use of XR capable wearable devices 102.

Therefore, according to some embodiments of the present disclosure, the wearable device 102 implements a method for controlling an operation of the wearable device 102. Further, the network node 108 implements a method for controlling an operation of the wearable device 102.

According to some embodiments of the present disclosure, the wearable device 102 identifies an activity status for user activity using the wearable device 102. For example, the activity performed by the user while wearing the wearable device 102 is identified by the wearable device 102. The wearable device provides information comprising at least one parameter associated with the wearable device 102 and the identified activity status. For example, the at least one parameter of the wearable device 102 may comprise one or more of a battery level of the wearable device 102, a position of the wearable device 102, a movement of the wearable device 102, an orientation of the wearable device 102, and one or more applications executing at the wearable device 102. In some embodiments, the wearable device 102 obtains the activity status and/or the at least one parameter associated with the wearable device from one or more sensors equipped in the wearable device 102. In some embodiments, the wearable device 102 determines the activity performed by the user based on the information obtained from the sensors. Further, the wearable device 102 transmits the provided information to the network node 108 in the wireless communication network. In some embodiments, the wearable device 102 directly transmits the data received from the sensors to the network node 108.

The wearable device 102 receives control information from the network node 108. The control information comprises at least one indication for controlling the operation of the wearable device 102. Further, the operation of the wearable device 102 is controlled in accordance with the control information received from the network node 108. For example, the operation of the wearable device 102 is changed in accordance with the control information.

According to some embodiments of the present disclosure, the network node 108 receives the information from the wearable device 102. The information comprises at least one parameter associated with the wearable device 102 and an activity status for user activity using the wearable device. In some embodiments, at least a part of the information is received from the one or more sensors equipped in the wearable device 102. The network node 108 provides control information for control of the wearable device 102. The control information is based on the information received from the wearable device 102. Further, the network node 108 transmits the provided control information to the wearable device 102. The control information comprises at least one indication for controlling an operation of the wearable device 102.

The operation of the wearable device 102 is controlled in accordance with the control information. The control information is transmitted by the network node 108 according to the information received from the wearable device 102. Thus, the operation of the wearable device 102 is controlled according to the at least one parameter of the wearable device 102 and the user activity with the wearable device 102. For example, when the user is watching video on the wearable device 102 and while wearing the wearable device 102 if the user starts some other activity. In such case, the network node 108 transmits control information indicating reduction of the resource allocation for the wearable device 102. The wearable device 102 changes the size and quality of the video on the screen of the wearable device 102 in accordance with the control information received from the network node 108. Therefore, the power utilization for displaying the video on the wearable device 102 may be reduced. Thereby, the embodiments herein provides optimization of the power consumption of the wearable device.

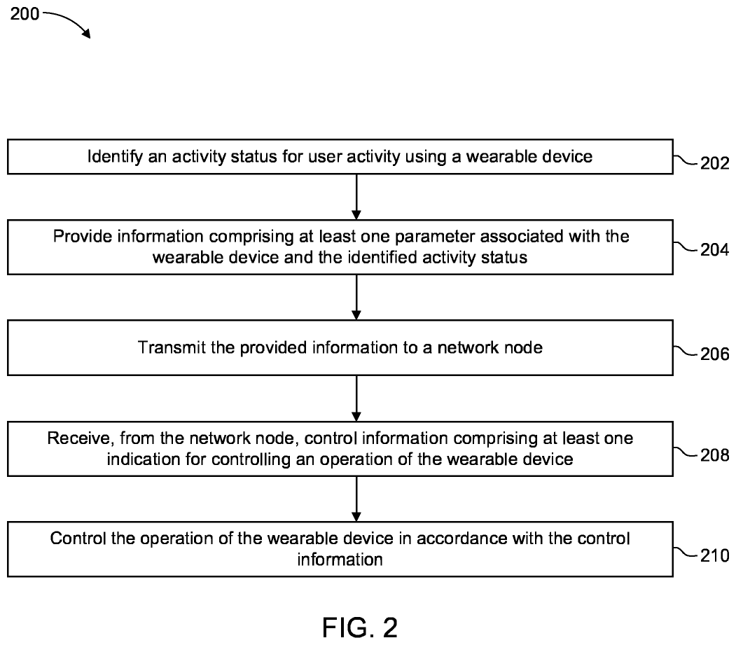

FIG. 2 is a flowchart illustrating example method steps of a method 200 performed by the wearable device for controlling an operation of the wearable device. The wearable device is connected to the network node in the wireless communication network.

At step 202, the method 200 comprises identifying an activity status for user activity using the wearable device. The activity status comprises one or more of information whether the wearable device being worn by the user or not, an eye gaze movement pattern of the user on a screen of the wearable device, a velocity with which the user is moving while wearing the wearable device, conversation of the user while wearing the wearable device, presence of another user in proximity to the user, and an eye gaze direction of the user on the wearable device.

In an example, the wearable device detects whether the user is wearing the wearable device or not. In another example, the wearable device determines the eye gaze movement pattern indicating a pattern of eyes of the user while watching a video on a screen of the wearable device. In another example, the wearable device determines a velocity with which the user is moving while wearing the wearable device. The wearable device further determines a state of the user according to the determined velocity. The state of the user includes a running state, a walking state, a driving state, or a sport training state. In another example, the wearable device detects conversation of the user while wearing the wearable device. For example, the wearable device obtains voice information of the user and determines whether the user is in conversation with another user or if the user is on a phone call based on the voice information. In another example, the wearable device detects presence of another user in proximity to the user. For example, the wearable device scans the surrounding of the user and detects whether another user is present adjacent to the user wearing the wearable device. In another example, the wearable device detects the eye gaze direction of the user on the wearable device. The wearable device determines a level of attention of the user on the wearable device on the basis of the eye gaze direction of the user on the wearable device.

In some embodiments, the wearable device identifies the activity status of the user using the wearable device. For example, the wearable device determines the user action with the wearable device using Artificial Intelligence, AI, algorithm or Machine Learning, ML, algorithm to determine the activity status.

At step 204, the method 200 comprises providing information comprising at least one parameter associated with the wearable device and the identified activity status. For example, the wearable device obtains the information and provides the information for determination of the activity status. The parameter associated with the wearable device comprises one or more of a battery level of the wearable device, a position of the wearable device, a movement of the wearable device, an orientation of the wearable device, and one or more applications executing at the wearable device. For example, the battery level of the wearable device includes one of a low battery level, a full battery level, or partially charged level. The position of the wearable device indicates a co-ordinate of the wearable device in real space. The movement of the wearable device indicates whether the wearable device is moving in the real space. The applications executing at the wearable device includes one or more of an augmented reality, AR application, a virtual reality, VR, application, a mixed reality, MR, application, or an extended reality, XR, application.

In an embodiment, the wearable device identifies the activity status for user activity using the wearable device and provides the information comprising at least one parameter associated with the wearable device and the identified activity status.

In another embodiment, the wearable device obtains the information from one or more sensors equipped in the wearable device. For example, the sensors includes one or more of a battery state sensor, a position sensor (e.g. Global Positioning System, GPS), an accelerometer, a gyroscope, and a proximity sensor. The sensor equipped in the wearable device detects the information comprising at least one parameter of the wearable device and the user activity with the wearable device. For example, the battery state sensor detects the battery level of the wearable device. The position sensor detects the position of the wearable device in the real environment. The accelerometer detects the movement of the wearable device. The gyroscope detects the orientation and angular velocity of the wearable device. The proximity sensor detects presence of another user in proximity to the user wearing the wearable device.

At step 206, the method 200 comprises transmitting the provided information to the network node in the wireless communication network. For example, the wearable device transmits the at least one parameter associated with the wearable device and the activity status of the user to the network node.

In some embodiments, the wearable device transmits the information obtained from the sensors to the network node. For example, the wearable device transmits the information obtained from the sensors, to the network node without processing of the information.

At step 208, the method 200 comprises receiving, from the network node, control information comprising at least one indication for controlling an operation of the wearable device. The control information instructs the wearable device to change the current operation being executed in the wearable device. Examples of the control information may comprise one or more of an indication to change a Radio Resource Control, RRC, state of the wearable device, a change in an allocation of radio resources for the wearable device, an indication to change in video encoding parameters associated with the wearable device in accordance with the activity status using the wearable device, an indication for monitoring of a Physical Downlink Control Channel, PDCCH, information, and an indication for a RRC connection reconfiguration of the wearable device.

In some examples, the control information instructs the wearable device to change the current RRC state to another RRC state. The RRC state may comprise a RRC connected state, a RRC idle state, a RRC connection release state, or the like. For example, when the wearable device is in RRC connected state and the user starts another activity (e.g. biking, running, driving, or the like), the wearable device may receive the control information comprising an indication to change the RRC connected state to the RRC idle state. Thus, the power consumption can be reduced in operating the wearable device.

In some examples, the control information indicates a change in the allocation of the radio resources for the wearable device. The network node may change the allocation of the radio resources for the wearable device according to the information received from the wearable device. For example, the network node may release the radio resources reserved for transmitting the content to the wearable device when the user activity is detected. The wearable device receives the control information indicating that the reserved radio resources have been released. Thus, the power consumption can be reduced in operating the wearable device. Further, when the user activity is completed and the user resumes the video then the network node again reserves the radio resources for transmitting the content to the wearable device.

In some examples, the control information comprises the indication to change video encoding parameters associated with the wearable device in accordance with in accordance with the activity status using the wearable device. For example, when the user is watching a video using the wearable device and based on the detected eye gaze movement of the user, the wearable device may receive the control information indicating the wearable device to change the video encoding parameters.

In some examples, the control information comprises micro sleep commands to allow the wearable device to avoid an air interface monitoring, i.e. avoiding reference signals reception or PDCCH monitoring.

In some examples, the control information comprises an indication for the RRC connection reconfiguration of the wearable device. For example, the control information instructs the wearable device to reconfigure the RRC connection of the wearable device according to the information comprising at least one parameter associated with the wearable device and the user activity using the wearable device.

At step 210, the method 200 comprises controlling the operation of the wearable device in accordance with the control information received from the network node. The wearable device changes the operation according to the control information. For example, the wearable device changes the current operation which consumes more power to another operation which consumes less power.

Thus, the wearable device controls the operation in accordance with the control information received from the network node. The control information is based on the information related to the wearable device and the user activity using the wearable device. Therefore, the operation of the wearable device is controlled based on the information related to the wearable device and the user activity using the wearable device. Thus, the usage of the radio resources and the power consumption of the wearable device may be optimized. Thereby, the embodiments herein provides optimization of the usage of the radio resources and power consumption of wearable devices.

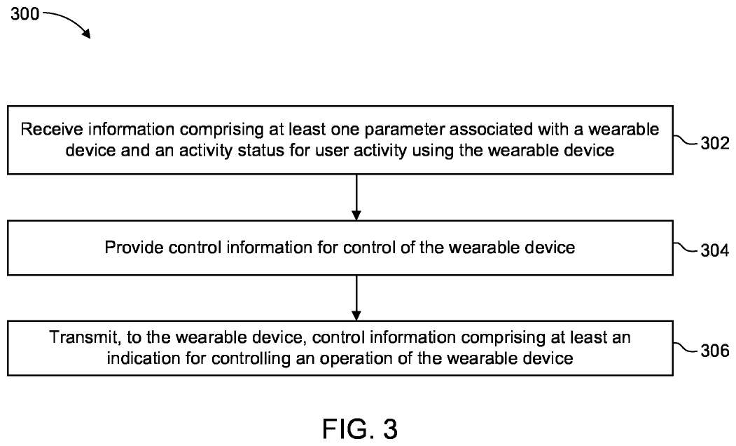

FIG. 3 is a flowchart illustrating example method steps of a method 300 performed by the network node for controlling an operation of the wearable device. The wearable device is connected to the network node in the wireless communication network.

At step 302, the method 300 comprises receiving, from the wearable device, information comprising at least one parameter associated with the wearable device and an activity status for user activity using the wearable device. For example, the parameter associated with the wearable device comprises one or more of a battery level of the wearable device, a position of the wearable device, a movement of the wearable device, an orientation of the wearable device, and one or more applications executing at the wearable device. For example, the user activity using the wearable device comprises one or more of information whether the wearable device being worn by the user or not, an eye gaze movement pattern of the user on a screen of the wearable device, a velocity with which the user is moving while wearing the wearable device, conversation of the user while wearing the wearable device, presence of another user in proximity to the user, and an eye gaze direction of the user on the wearable device.

In an embodiment, the network node receives the information from the wearable device. For example, the information is processed by the wearable device using the AI algorithm or ML algorithm to identify one or more parameters and the activity status of the user activity. The identified one or more parameters and the activity status is received from the wearable device.

In another embodiment, at least a part of the information is received directly from the one or more sensors equipped in the wearable device. For example, the network node receives the information directly from the sensors, without processing of the information at the wearable device. The network node processes at least the part of the information to determine the at least one parameter associated with the wearable device and the activity status for the user activity.

At step 304, the method comprises providing control information for control of the wearable device. The control information is based on the information received from the wearable device. For example, the network node determines or generates the control information based on the information received from the wearable device. For example, the control information comprises at least the indication for controlling the operation of the wearable device. In another example, the control information indicates the wearable device to change the currently executing operation. Examples of the control information may comprise one or more of an indication to change the RRC, state of the wearable device, a change in an allocation of radio resources for the wearable device, an indication to change video encoding parameters associated with the wearable device in accordance with the activity status, an indication for monitoring of the PDCCH information, and an indication for a RRC connection reconfiguration of the wearable device.

At step 306, the method 300 comprises transmitting, to the wearable device, the provided control information comprising at least one indication for controlling an operation of the wearable device.

The control information is transmitted to the wearable device for controlling the operation of the wearable device. Therefore, the operation of the wearable device is being controlled based on the information related to the wearable device and the user activity using the wearable device. Thus, the usage of the radio resources along with the power consumption at the wearable device may be optimized. Thereby, the embodiments herein provides optimization of the usage of the radio resources along with the power consumption at the wearable device.

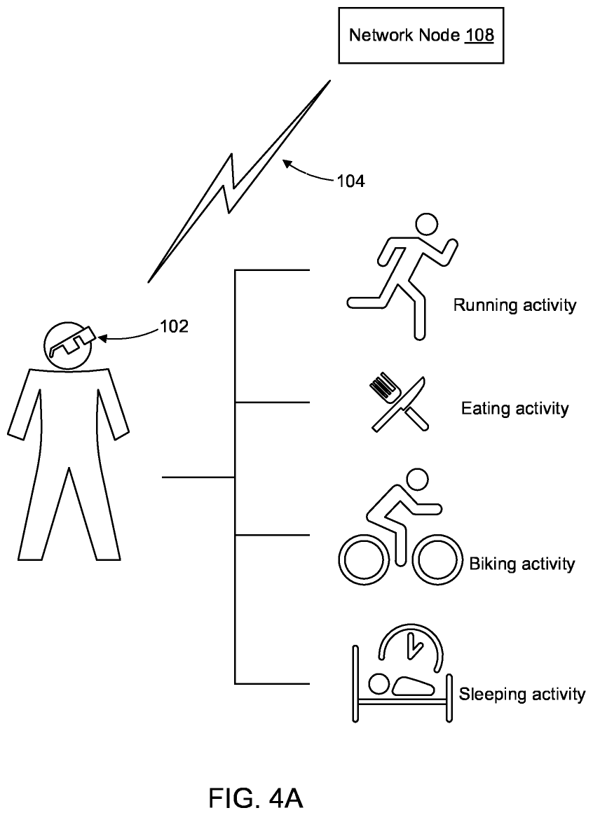

FIG. 4A discloses an example of user activity using the wearable device. As depicted in FIG. 4A, the user is wearing the wearable device 102. The wearable device 102 is connected to the network node 108 through the wireless link 104. The user may watch a video content on a screen of the wearable device. The video content is being received from the network node 108 over the radio resources allocated to the wearable device. Further, the user starts an activity while wearing the wearable device 102. The activity of the user may include one or more of running, eating, biking, sleeping, or the like (as illustrated in FIG. 4A). The user activity is identified by the wearable device 102. In one example, the wearable device 102 identifies that the user starts biking while wearing the wearable device 102. The wearable device 102 transmits the information to the network node 108 with an indication that the user is in biking state while wearing the wearable device 102. The network node 108 receives the information from the wearable device 102 and the network node 108 may allocate less radio resource for the transmission of the video content as user is in mobility due to the biking activity of the user. The network node 108 further transmits control information to the wearable device 102 to indicate that the radio resources for the video content has been reduced for the transmission of the video content. For example, the wearable device 102 may changes quality/size of the video content on the screen of the wearable device 102 such that the user is able to focus of the road. Thus, the wearable device 102 controls the operation of the wearable device in accordance with the control information. In some examples, the wearable device 102 may reduce the monitoring of the PDCCH information based on the control information. Thus, by changing the quality/size of the video content and reducing the monitoring of the PDCCH information, the wearable device 102 is allowed to optimize the usage of the radio resources along with the power consumption at the wearable device 102.

FIG. 4B discloses an example illustration of displaying a content 402 in field of view, FoV of the wearable device 102. The wearable device 102 is connected to the network node 108 through the wireless link 104. As depicted in FIG. 4B, the user is watching the content 402 on a screen 404 of the wearable device 102. The wearable device 102 identifies that an eye gaze of the user in at position 406 on the screen 404 of the wearable device 102 while watching the content. Further, the wearable device 102 identifies a movement of the eye gaze of the user from the position 406 to a position 408. Accordingly, the wearable device 102 may determine that the user's eye gaze movement is shifted from the position 406 to position 408 on the screen 404. The wearable device 102 identifies that the user is not interested in the content being rendered at the position 406 on the screen. Further, the wearable device 102 transmits the information indicating the eye gaze movement to the network node 108. The network node 108 provides the control information indicating a change in video encoding parameters for the content being rendered in the wearable device 102. Further, the network node 108 transmits the control information to the wearable device 102 for controlling the operation of the wearable device 102. Further, the wearable device 102 receives control information from the network node 108 indicating the change in video encoding parameters and the wearable device 102 adapts the encoding parameters for rendering the content at the position 406 on the screen 404 of the wearable device 102. Thus, by changing the video encoding parameters, the wearable device 102 is allowed to optimize the usage of the radio resources along with the power consumption at the wearable device 102.

FIG. 4C discloses an example illustration of displaying a content 402 in field of view, FoV of the wearable device 102. The wearable device 102 is connected to the network node 108 through a wireless link 104. As depicted in FIG. 4C, the user is watching the content 402 on a screen 404 of the wearable device 102. Further, the wearable device 102 identifies that the user partially removed the wearable device 102 from his eyes. The wearable device 102 transmits the information to the network node 108 indicating that the user has partially removed the wearable device 102. Further, the wearable device 102 receives control information comprising an indication to change a RRC state of the wearable device 102. Further, the wearable device 102 changes the RRC state from a RRC connected state to a RRC idle state. Thus, by changing the RRC states of the wearable device 102, the wearable device 102 is allowed to optimize the usage of the radio resources along with the power consumption at the wearable device 102.

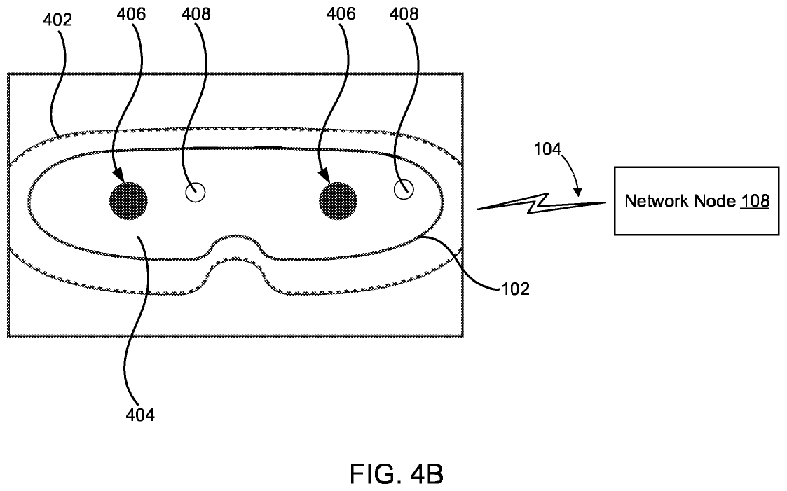

FIG. 5 is an example schematic diagram illustrating the wearable device 102 and apparatus 108. The apparatus 108 may e.g. be comprised in a network node. The wearable device 102 is configured to cause performance of the method 200 for controlling an operation of the wearable device. The apparatus 108 is configured to cause performance of the method 300 for controlling the operation of the wearable device. The apparatus 102 is connected to the network node 108.

According to at least some embodiments of the present invention, the apparatus 102 in FIG. 5 comprises one or more modules. These modules may e.g. be sensors 502, an operating system 504, a controlling circuitry 506, a processor 508, a user activity identifier 510 and a transceiver 512.

As described above, the various ways of controlling the operation of the wearable device, a few of which have been mentioned above in connection to the explanation of FIG. 2.

The sensors 502 are configured to provide at least one parameter associated with the wearable device. Examples of the sensors may comprise one or more of the battery state sensor, the position sensor (e.g. GPS), the accelerometer, the gyroscope, the proximity sensor, or the like.

The user activity identifier 510 is configured to identify the activity status for user activity using the wearable device.

The transceiver 510 is configured to transmit the information provided by the operating system 504 to the network node 108. Further, the transceiver 510 is configured to receive the control information from the network node 108.

The operating system 504 is configured to control the operation of the wearable device 102 in accordance with the control information received from the network node 108. For example, the operating system may change the current operation of the wearable device to another operation on the basis of the control information received from the network node 108.

The controlling circuitry 506, may in some embodiments be adapted to control the above mentioned modules.

The sensors 502, the operating system 504, the processor 508, the user activity identifier 510, and the transceiver 510 as well as the controlling circuitry 506, may be operatively connected to each other.

According to at least some embodiments of the present invention, the apparatus 108 in FIG. 5 comprises one or more modules. These modules may e.g. be an analyser 522, an optimizer 520, a controlling circuitry 518, a processor 516, and a transceiver 514. As described above, the various ways of controlling the operation of the wearable device, a few of which have been mentioned above in connection to the explanation of FIG. 3.

The transceiver 514 is configured to receive the information comprising at least one parameter associated with the wearable device 102 and an activity status for user activity using the wearable device 102. In another embodiment, the information is received directly from the sensors 502 equipped in the wearable device 102. Further, the transceiver 514 is configured to transmit the control information to the wearable device 102.

The analyser 522 is configured to analyse the activity status for user activity according to the information received from the wearable device 102. In some embodiments, the analyser is configured to analyse the information received from one or more sensors equipped in the wearable device. For example, the information received from one or more sensors is analyzed by the analyser 522 to identify the activity status for user activity.

The optimizer 520 is configured to provide the control information in order to optimize the power consumption of the wearable device 102 and also the usage of the radio resources for the wearable device 102. In an embodiment, the optimizer 520 may be equipped in the network node 108. In another embodiment, the optimizer 520 may be present outside the network node 108. For example, the optimizer 520 may be present in the cloud or in a core node.

The controlling circuitry 518, may in some embodiments be adapted to control the above mentioned modules.

The analyser 522, the optimizer 520, the processor 516, and the transceiver 514 as well as the controlling circuitry 518, may be operatively connected to each other.

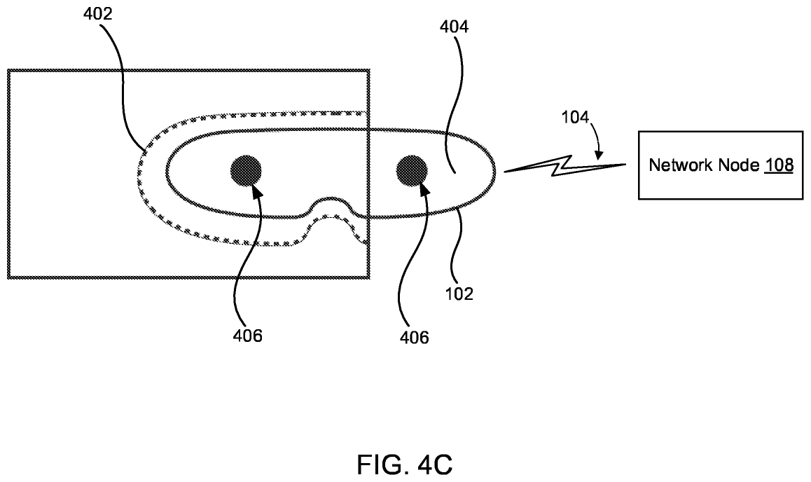

FIG. 6 illustrates an example computing environment 600 implementing a method and the network node and the UE as described in FIGS. 2 and 3. As depicted in FIG. 6, the computing environment 600 comprises at least one processing unit 602 that is equipped with a control unit 604 and an Arithmetic Logic Unit (ALU) 606, a plurality of networking devices 608 and a plurality Input output, I/O devices 610, a memory 612, and a storage 614. The processing unit 602 may be responsible for implementing the method described in FIGS. 2 and 3. For example, the processing unit 602 may in some embodiments be equivalent to the processor of the network node and the wearable device as described above in conjunction with the FIGS. 1-5. The processing unit 602 is capable of executing software instructions stored in memory 612. The processing unit 602 receives commands from the control unit 604 in order to perform its processing. Further, any logical and arithmetic operations involved in the execution of the instructions are computed with the help of the ALU 606.

The computer program is loadable into the processing unit 602, which may, for example, be comprised in an electronic apparatus (such as a UE or a network node). When loaded into the processing unit 602, the computer program may be stored in the memory 612 associated with or comprised in the processing unit 602. According to some embodiments, the computer program may, when loaded into and run by the processing unit 602, cause execution of method steps according to, for example, any of the methods illustrated in FIGS. 2 and 3 or otherwise described herein.

The overall computing environment 600 may be composed of multiple homogeneous and/or heterogeneous cores, multiple CPUs of different kinds, special media and other accelerators. Further, the plurality of processing unit 602 may be located on a single chip or over multiple chips.

The algorithm comprising of instructions and codes required for the implementation are stored in either the memory 612 or the storage 614 or both. At the time of execution, the instructions may be fetched from the corresponding memory 612 and/or storage 614, and executed by the processing unit 602.

In case of any hardware implementations various networking devices 608 or external I/O devices 610 may be connected to the computing environment to support the implementation through the networking devices 608 and the I/O devices 610.

The embodiments disclosed herein can be implemented through at least one software program running on at least one hardware device and performing network management functions to control the elements. The elements shown in FIG. 6 include blocks which can be at least one of a hardware device, or a combination of hardware device and software module.

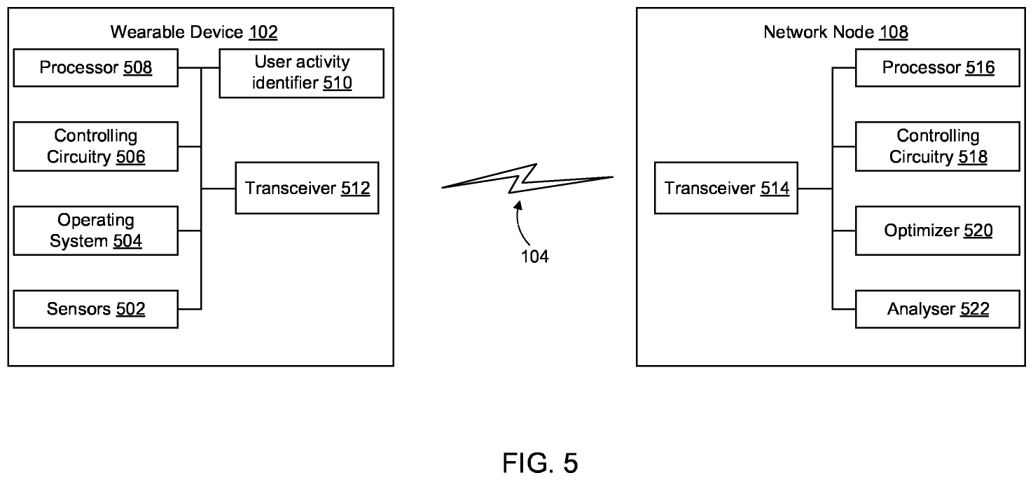

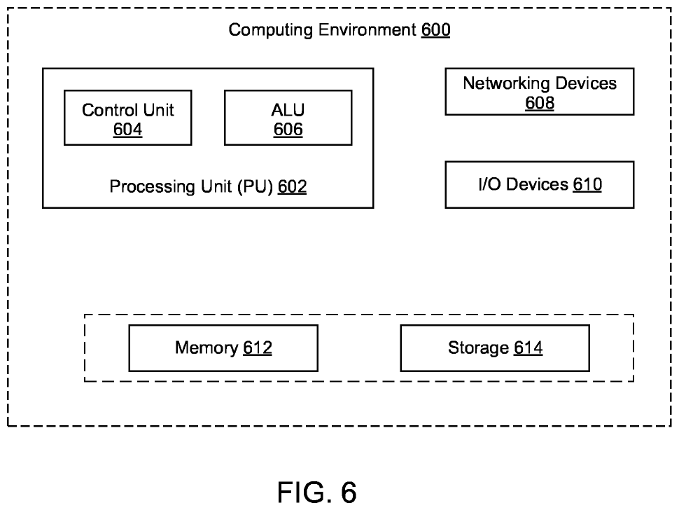

FIG. 7 is a block diagram of a telecommunication network connected via an intermediate network to a host computer according to some embodiments. With reference to FIG. 8, in accordance with an embodiment, a communication system includes telecommunication network 4410, such as a 3GPP-type cellular network, which comprises access network 4411, such as a radio access network, and core network 4414. Access network 4411 comprises a plurality of base stations 4412a, 4412b, 4412c, such as NBs, eNBs, gNBs or other types of wireless access points, each defining a corresponding coverage area 4413a, 4413b, 4413c. Each base station 4412a, 4412b, 4412c is connectable to core network 4414 over a wired or wireless connection 4415. A first UE 4491 located in coverage area 4413c is configured to wirelessly connect to, or be paged by, the corresponding base station 4412c. A second UE 4492 in coverage area 4413a is wirelessly connectable to the corresponding base station 4412a. While a plurality of UEs 4491, 4492 are illustrated in this example, the disclosed embodiments are equally applicable to a situation where a sole UE is in the coverage area or where a sole UE is connecting to the corresponding base station 4412.

Telecommunication network 4410 is itself connected to host computer 4430, which may be embodied in the hardware and/or software of a standalone server, a cloud-implemented server, a distributed server or as processing resources in a server farm. Host computer 4430 may be under the ownership or control of a service provider, or may be operated by the service provider or on behalf of the service provider. Connections 4421 and 4422 between telecommunication network 4410 and host computer 4430 may extend directly from core network 4414 to host computer 4430 or may go via an optional intermediate network 4420. Intermediate network 4420 may be one of, or a combination of more than one of, a public, private or hosted network; intermediate network 4420, if any, may be a backbone network or the Internet; in particular, intermediate network 4420 may comprise two or more sub-networks (not shown).

The communication system of FIG. 7 as a whole enables connectivity between the connected UEs 4491, 4492 and host computer 4430. The connectivity may be described as an over-the-top, OTT connection 4450. Host computer 4430 and the connected UEs 4491, 4492 are configured to communicate data and/or signaling via OTT connection 4450, using access network 4411, core network 4414, any intermediate network 4420 and possible further infrastructure (not shown) as intermediaries. OTT connection 4450 may be transparent in the sense that the participating communication devices through which OTT connection 4450 passes are unaware of routing of uplink and downlink communications. For example, base station 4412 may not or need not be informed about the past routing of an incoming downlink communication with data originating from host computer 4430 to be forwarded (e.g., handed over) to a connected UE 4491. Similarly, base station 4412 need not be aware of the future routing of an outgoing uplink communication originating from the UE 4491 towards the host computer 4430.

FIG. 8 is a block diagram of a host computer communicating via a base station with a user equipment over a partially wireless connection. Example implementations, in accordance with an embodiment, of the UE, base station and host computer discussed in the preceding paragraphs will now be described with reference to FIG. 8. In communication system 4500, host computer 4510 comprises hardware 4515 including communication interface 4516 configured to set up and maintain a wired or wireless connection with an interface of a different communication device of communication system 4500. Host computer 4510 further comprises processing circuitry 4518, which may have storage and/or processing capabilities.

In particular, processing circuitry 4518 may comprise one or more programmable processors, application-specific integrated circuits, field programmable gate arrays or combinations of these (not shown) adapted to execute instructions. Host computer 4510 further comprises software 4511, which is stored in or accessible by host computer 4510 and executable by processing circuitry 4518. Software 4511 includes host application 4512. Host application 4512 may be operable to provide a service to a remote user, such as UE 4530 connecting via OTT connection 4550 terminating at UE 4530 and host computer 4510. In providing the service to the remote user, host application 4512 may provide user data which is transmitted using OTT connection 4550.

Communication system 4500 further includes base station 4520 provided in a telecommunication system and comprising hardware 4525 enabling it to communicate with host computer 4510 and with UE 4530. Hardware 4525 may include communication interface 4526 for setting up and maintaining a wired or wireless connection with an interface of a different communication device of communication system 4500, as well as radio interface 4527 for setting up and maintaining at least wireless connection 4570 with UE 4530 located in a coverage area (not shown in FIG. 7) served by base station 4520. Communication interface 4526 may be configured to facilitate connection 4560 to host computer 4510. Connection 4560 may be direct or it may pass through a core network (not shown in FIG. 8) of the telecommunication system and/or through one or more intermediate networks outside the telecommunication system. In the embodiment shown, hardware 4525 of base station 4520 further includes processing circuitry 4528, which may comprise one or more programmable processors, application-specific integrated circuits, field programmable gate arrays or combinations of these (not shown) adapted to execute instructions. Base station 4520 further has software 4521 stored internally or accessible via an external connection.

Communication system 4500 further includes UE 4530 already referred to. Its hardware 4535 may include radio interface 4537 configured to set up and maintain wireless connection 4570 with a base station serving a coverage area in which UE 4530 is currently located. Hardware 4535 of UE 4530 further includes processing circuitry 4538, which may comprise one or more programmable processors, application-specific integrated circuits, field programmable gate arrays or combinations of these (not shown) adapted to execute instructions. UE 4530 further comprises software 4531, which is stored in or accessible by UE 4530 and executable by processing circuitry 4538. Software 4531 includes client application 4532. Client application 4532 may be operable to provide a service to a human or non-human user via UE 4530, with the support of host computer 4510. In host computer 4510, an executing host application 4512 may communicate with the executing client application 4532 via OTT connection 4550 terminating at UE 4530 and host computer 4510. In providing the service to the user, client application 4532 may receive request data from host application 4512 and provide user data in response to the request data. OTT connection 4550 may transfer both the request data and the user data. Client application 4532 may interact with the user to generate the user data that it provides.

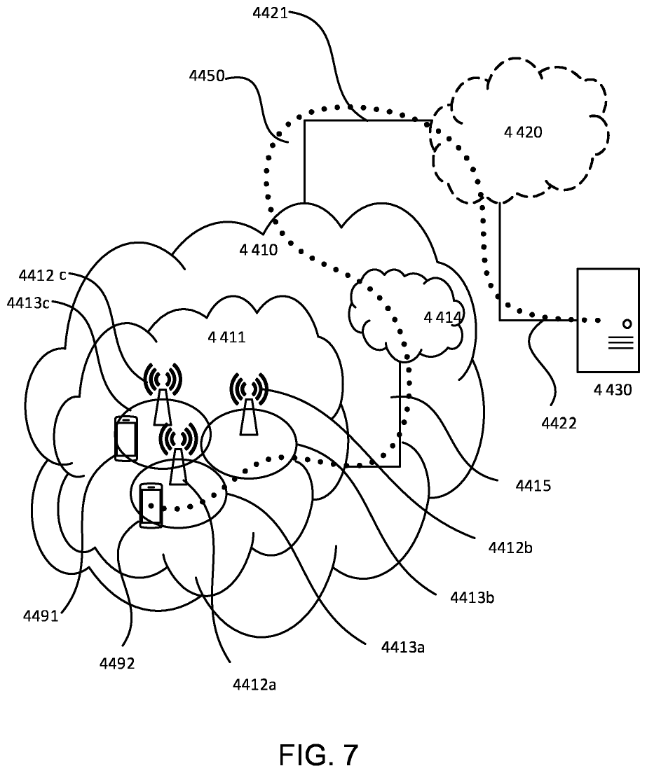

It is noted that host computer 4510, base station 4520 and UE 4530 illustrated in FIG. 7 may be similar or identical to host computer 4430, one of base stations 4412a, 4412b, 4412c and one of UEs 4491, 4492 respectively. This is to say, the inner workings of these entities may be as shown in FIG. 9 and independently, the surrounding network topology may be that of FIG. 8.

In FIG. 8, OTT connection 4550 has been drawn abstractly to illustrate the communication between host computer 4510 and UE 4530 via base station 4520, without explicit reference to any intermediary devices and the precise routing of messages via these devices. Network infrastructure may determine the routing, which it may be configured to hide from UE 4530 or from the service provider operating host computer 4510, or both. While OTT connection 4550 is active, the network infrastructure may further take decisions by which it dynamically changes the routing (e.g., on the basis of load balancing consideration or reconfiguration of the network).

Wireless connection 4570 between UE 4530 and base station 4520 is in accordance with the teachings of the embodiments described throughout this disclosure. One or more of the various embodiments may improve the performance of OTT services provided to UE 4530 using OTT connection 4550, in which wireless connection 4570 forms the last segment. More precisely, the teachings of these embodiments may improve the random access speed and/or reduce random access failure rates and thereby provide benefits such as faster and/or more reliable random access.

A measurement procedure may be provided for the purpose of monitoring data rate, latency and other factors on which the one or more embodiments improve. There may further be an optional network functionality for reconfiguring OTT connection 4550 between host computer 4510 and UE 4530, in response to variations in the measurement results. The measurement procedure and/or the network functionality for reconfiguring OTT connection 4550 may be implemented in software 4511 and hardware 4515 of host computer 4510 or in software 4531 and hardware 4535 of UE 4530, or both. In embodiments, sensors (not shown) may be deployed in or in association with communication devices through which OTT connection 4550 passes; the sensors may participate in the measurement procedure by supplying values of the monitored quantities exemplified above, or supplying values of other physical quantities from which software 4511, 4531 may compute or estimate the monitored quantities. The reconfiguring of OTT connection 4550 may include message format, retransmission settings, preferred routing etc.; the reconfiguring need not affect base station 4520, and it may be unknown or imperceptible to base station 4520. Such procedures and functionalities may be known and practiced in the art. In certain embodiments, measurements may involve proprietary UE signaling facilitating host computer 4510's measurements of throughput, propagation times, latency and the like. The measurements may be implemented in that software 4511 and 4531 causes messages to be transmitted, in particular empty or ‘dummy’ messages, using OTT connection 4550 while it monitors propagation times, errors or the like.

FIG. 9 is a block diagram of methods implemented in a communication system including a host computer, a base station, and a user equipment according to some embodiments. For simplicity of the present disclosure, only drawing references to FIG. 9 will be included in this section. In step 4610, the host computer provides user data. In substep 4611 (which may be optional) of step 4610, the host computer provides the user data by executing a host application. In step 4620, the host computer initiates a transmission carrying the user data to the UE. In step 4630 (which may be optional), the base station transmits to the UE the user data which was carried in the transmission that the host computer initiated, in accordance with the teachings of the embodiments described throughout this disclosure. In step 4640 (which may also be optional), the UE executes a client application associated with the host application executed by the host computer.

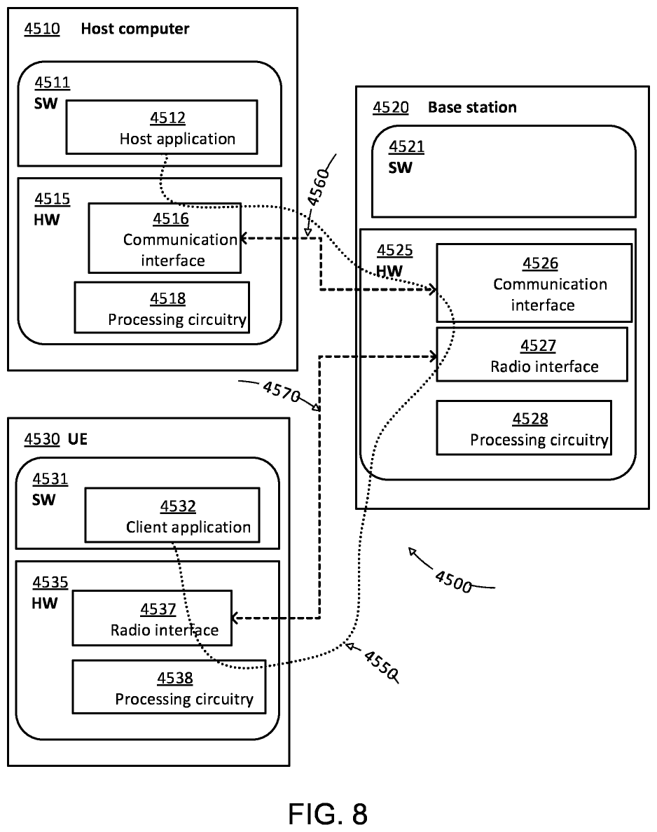

FIG. 10 is a block diagram of methods implemented in a communication system including a host computer, a base station, and a user equipment according to some embodiments. For simplicity of the present disclosure, only drawing references to FIG. 10 will be included in this section. In step 4710 of the method, the host computer provides user data. In an optional substep (not shown) the host computer provides the user data by executing a host application. In step 4720, the host computer initiates a transmission carrying the user data to the UE. The transmission may pass via the base station, in accordance with the teachings of the embodiments described throughout this disclosure. In step 4730 (which may be optional), the UE receives the user data carried in the transmission.

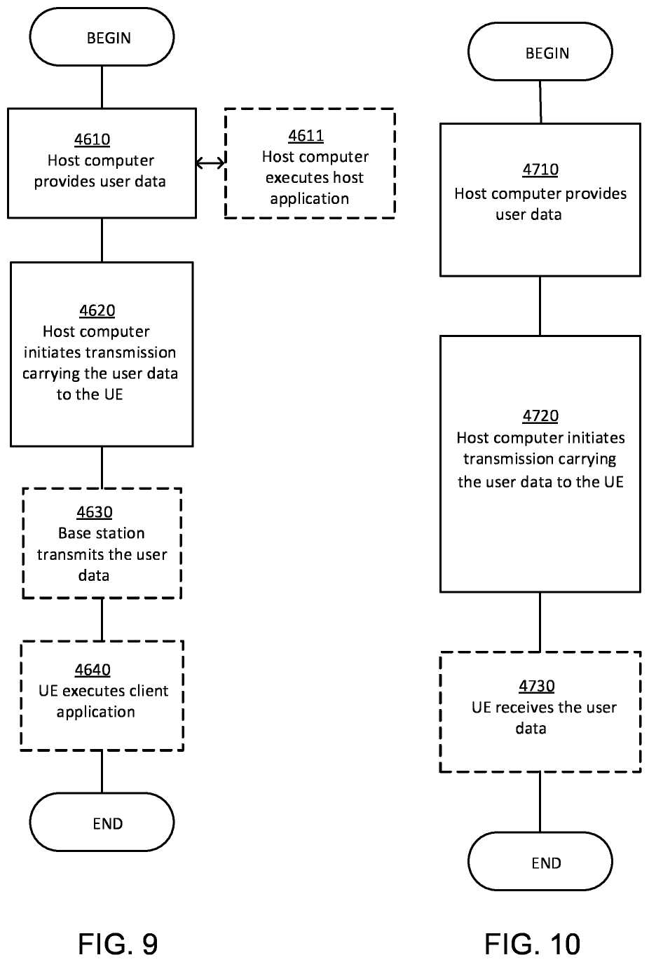

FIG. 11 is a block diagram of methods implemented in a communication system including a host computer, a base station, and a user equipment according to some embodiments. For simplicity of the present disclosure, only drawing references to FIG. 11 will be included in this section. In step 4810 (which may be optional), the UE receives input data provided by the host computer. Additionally or alternatively, in step 4820, the UE provides user data. In substep 4821 (which may be optional) of step 4820, the UE provides the user data by executing a client application. In substep 4811 (which may be optional) of step 4810, the UE executes a client application which provides the user data in reaction to the received input data provided by the host computer. In providing the user data, the executed client application may further consider user input received from the user. Regardless of the specific manner in which the user data was provided, the UE initiates, in substep 4830 (which may be optional), transmission of the user data to the host computer. In step 4840 of the method, the host computer receives the user data transmitted from the UE, in accordance with the teachings of the embodiments described throughout this disclosure.

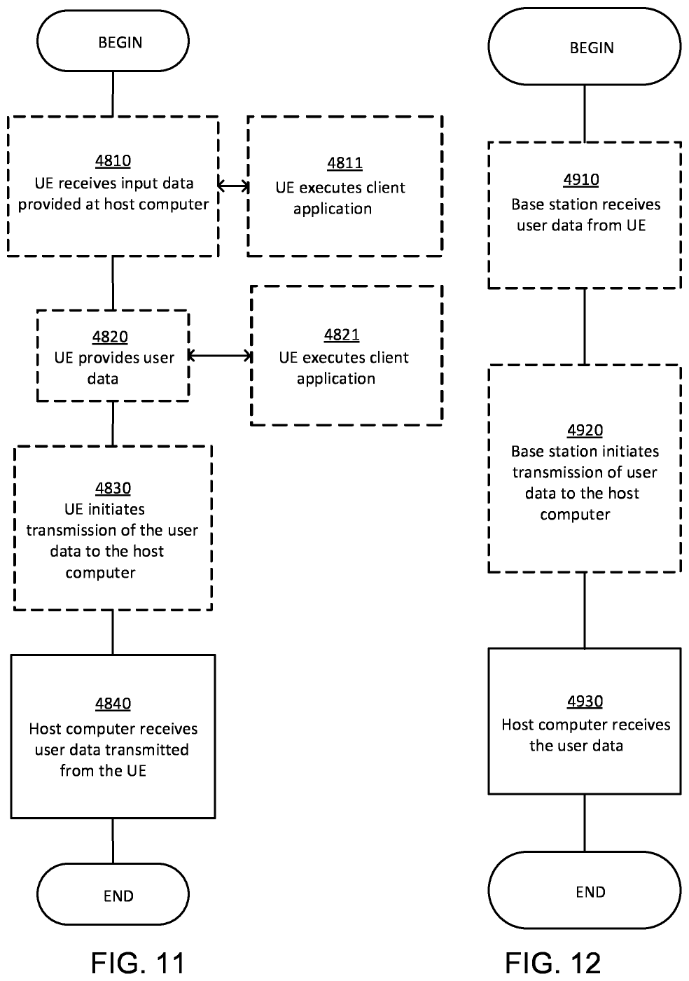

FIG. 12 is a block diagram of methods implemented in a communication system including a host computer, a base station, and a user equipment according to some embodiments. FIG. 12 is a flowchart illustrating a method implemented in a communication system, in accordance with one embodiment. The communication system includes a host computer, a base station and a UE. For simplicity of the present disclosure, only drawing references to FIG. 12 will be included in this section. In step 4910 (which may be optional), in accordance with the teachings of the embodiments described throughout this disclosure, the base station receives user data from the UE. In step 4920 (which may be optional), the base station initiates transmission of the received user data to the host computer. In step 4930 (which may be optional), the host computer receives the user data carried in the transmission initiated by the base station.

Any appropriate steps, methods, features, functions, or benefits disclosed herein may be performed through one or more functional units or modules of one or more virtual apparatuses. Each virtual apparatus may comprise a number of these functional units. These functional units may be implemented via processing circuitry, which may include one or more microprocessor or microcontrollers, as well as other digital hardware, which may include digital signal processors, DSPs, special-purpose digital logic, and the like. The processing circuitry may be configured to execute program code stored in memory, which may include one or several types of memory such as read-only memory (ROM), random-access memory, RAM, cache memory, flash memory devices, optical storage devices, etc. Program code stored in memory includes program instructions for executing one or more telecommunications and/or data communications protocols as well as instructions for carrying out one or more of the techniques described herein. In some implementations, the processing circuitry may be used to cause the respective functional unit to perform corresponding functions according one or more embodiments of the present disclosure.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the disclosure.

1. A method for controlling an operation of a wearable device, the wearable device being connected to a network node in a wireless communication network, the method being performed by the wearable device, the method comprising:
identifying an activity status for user activity using the wearable device;
providing information comprising at least one parameter associated with the wearable device and the identified activity status;
transmitting the provided information to the network node in the wireless communication network;
receiving, from the network node, control information comprising at least one indication for controlling the operation of the wearable device; and
controlling the operation of the wearable device in accordance with the control information received from the network node.

2. The method according to claim 1, further comprising:
obtaining the activity status and/or the at least one parameter associated with the wearable device from one or more sensors equipped in the wearable device.

3. The method according to claim 1, wherein the at least one parameter associated with the wearable device comprises one or more of:
a battery level of the wearable device;
a position of the wearable device;
a movement of the wearable device;
an orientation of the wearable device; and
one or more applications executing at the wearable device.

4. The method according to claim 1, wherein the activity status comprises one or more of:
information whether the wearable device being worn by the user or not;
an eye gaze movement pattern of the user on a screen of the wearable device;
a velocity with which the user is moving while wearing the wearable device;
conversation of the user while wearing the wearable device;
presence of another user in proximity to the user; and
an eye gaze direction of the user on the wearable device.

5. The method according to claim 1, wherein the control information comprises one or more of:
an indication to change a Radio Resource Control (RRC) state of the wearable device;
a change in an allocation of radio resources for the wearable device;
an indication to change in video encoding parameters associated with the wearable device in accordance with the activity status using the wearable device;
an indication for monitoring of a Physical Downlink Control Channel (PDCCH) information; and
an indication for a RRC connection reconfiguration of the wearable device.

6. A method for controlling an operation of a wearable device, the wearable device being connected to a network node in a wireless communication network, the method being performed by the network node, the method comprising:
receiving, from the wearable device, information comprising at least one parameter associated with the wearable device and an activity status for user activity using the wearable device;
providing control information for control of the wearable device, the control information being based on the information received from the wearable device; and
transmitting, to the wearable device, the provided control information comprising at least one indication for controlling the operation of the wearable device.

7. The method according to claim 6, wherein at least a part of the information is received from one or more sensors equipped in the wearable device.

8. The method according to claim 6, wherein the at least one parameter associated with the wearable device comprises one or more of:
a battery level of the wearable device;
a position of the wearable device;
a movement of the wearable device;
an orientation of the wearable device; and
one or more applications executing at the wearable device.

9. The method according to claim 6, wherein the activity status comprises one or more of:
information whether the wearable device being worn by the user or not;
an eye gaze movement pattern of the user on a screen of the wearable device;
a velocity with which the user is moving while wearing the wearable device;
conversation of the user while wearing the wearable device;
presence of another user in proximity to the user; and
an eye gaze direction of the user on the wearable device.

10. The method according to claim 6, wherein the control information comprises one or more of:
an indication to change a Radio Resource Control (RRC) state of the wearable device;
a change in an allocation of radio resources for the wearable device;
an indication to change in video encoding parameters associated with the wearable device in accordance with the activity status using the wearable device;
an indication for monitoring of a Physical Downlink Control Channel (PDCCH) information; and
an indication for a RRC connection reconfiguration of the wearable device.

11. A wearable device for controlling an operation of the wearable device, the wearable device being connected to a network node in a wireless communication network, the wearable device comprising controlling circuitry configured to cause:
identification of an activity status for user activity using the wearable device;
providing of information comprising at least one parameter associated with the wearable device and the identified activity status;
transmission of the provided information to the network node in the wireless communication network;
reception of control information from the network node, the control information comprising at least one indication for controlling the operation of the wearable device; and
controlling of the operation of the wearable device in accordance with the control information received from the network node.

12. The wearable device according to claim 11, wherein the controlling circuitry is further configured to cause:
obtaining of the activity status and/or the at least one parameter associated with the wearable device from one or more sensors equipped in the wearable device.

13. The wearable device according to claim 11, wherein the at least one parameter associated with the wearable device comprises one or more of:
a battery level of the wearable device;
a position of the wearable device;
a movement of the wearable device;
an orientation of the wearable device; and
one or more applications executing at the wearable device.

14. The wearable device according to claim 11, wherein the activity status comprises one or more of:
information whether the wearable device being worn by the user or not;
an eye gaze movement pattern of the user on a screen of the wearable device;
a velocity with which the user is moving while wearing the wearable device;
conversation of the user while wearing the wearable device;
presence of another user in proximity to the user; and
an eye gaze direction of the user on the wearable device.

15. The wearable device according to claim 11, wherein the control information comprises one or more of:
an indication to change a Radio Resource Control (RRC) state of the wearable device;
a change in an allocation of radio resources for the wearable device;
an indication to change in video encoding parameters associated with the wearable device in accordance with the activity status using the wearable device;
an indication for monitoring of a Physical Downlink Control Channel (PDCCH) information; and
an indication for a RRC connection reconfiguration of the wearable device.

16. An apparatus of a network node configured to operate in a wireless communication network for controlling an operation of a wearable device, the wearable device being connected to the network node in the wireless communication network, the apparatus comprising controlling circuitry configured to cause:
reception of information from the wearable device, the information comprising at least one parameter associated with the wearable device and an activity status for user activity using the wearable device;
providing of control information for control of the wearable device, the control information being based on the information received from the wearable device; and
transmission of the provided control information to the wearable device, the provided control information comprising at least one indication for controlling the operation of the wearable device.

17. The apparatus according to claim 16, wherein at least a part of the information is received from one or more sensors equipped in the wearable device.

18. The apparatus according to claim 16, wherein the at least one parameter associated with the wearable device comprises one or more of:
a battery level of the wearable device;
a position of the wearable device;
a movement of the wearable device;
an orientation of the wearable device; and
one or more applications executing at the wearable device.

19. The apparatus according to claim 16, wherein the activity status comprises one or more of:
information whether the wearable device being worn by the user or not;
an eye gaze movement pattern of the user on a screen of the wearable device;
a velocity with which the user is moving while wearing the wearable device;
conversation of the user while wearing the wearable device;
presence of another user in proximity to the user; and
an eye gaze direction of the user on the wearable device.

20. The apparatus according to claim 16, wherein the control information comprises one or more of:
an indication to change a Radio Resource Control (RRC) state of the wearable device;
a change in an allocation of radio resources for the wearable device;
an indication to change in video encoding parameters associated with the wearable device in accordance with the activity status using the wearable device;
an indication for monitoring of a Physical Downlink Control Channel (PDDCH) information; and
an indication for a RRC connection reconfiguration of the wearable device.

21-22. (canceled)
